(19) United States
(12) Patent Application Publication
(10) Pub. No.: US 2008/0255062 A1
(43) Pub. Date: Oct. 16, 2008

(54) AXON REGENERATION FROM ADULT SENSORY NEURONS

(75) Inventor: Paul FERNYHOUGH, Winnipeg (CA)

Correspondence Address: FASKEN MARTINEAU DUMOULIN, LLP, 2900 - 550 Burrard Street, VANCOUVER, BC, V6C 0A3, CA

(73) Assignee: UNIVERSITY OF MANITOBA, Winnipeg (CA)

(21) Appl. No.: 12/030,493

(22) Filed: Feb. 13, 2008

Related U.S. Application Data
(60) Provisional application No. 60/889,693, filed on Feb. 13, 2007.

(51) Int. Cl. A61K 31/7042; C12N 5/06; C12Q 1/02; C12M 3/00; A61K 31/426; A61K 31/192; A61K 31/164; A61K 31/19; A61K 31/195; A61K 31/437

(52) U.S. Cl. 514/37; 435/325; 435/29; 435/288.4; 514/567; 514/299; 514/557; 514/368; 514/620; 514/570

(57) ABSTRACT

A method for rapidly screening small molecules to identify small molecules that stimulate axon regeneration and outgrowth from adult sensory neurons. The method generally comprises preparing a purified individualized dorsal root ganglia cell suspension (DRG), coating well surfaces of a suitably prepared multi-well microplate with the DRG, then dispensing dosages of selected small molecules into selected wells. The microplates are incubated under sterile conditions at about 37° C. for at least 24 hours. The DRG suspension in each well is then morphometrically assessed to assess the extent of axon regeneration and outgrowth that occurred, and the effects of the selected small molecules are determined by comparison to control treatments. The method is suitable for screening chemically derived small molecules and biologically derived small molecules.

### FIELD OF THE INVENTION

This invention relates to screening of molecules for their efficacy on physiological processes. More particularly, this invention relates to methods, apparatus and kits for screening of molecules useful for stimulating axon regeneration from adult somatic neurons.

### BACKGROUND OF THE INVENTION

Sensory neurons are nerve cells within the nervous system responsible for converting external stimuli from an organism's environment into internal electrical motor reflex, and therefore, are considered part of the peripheral nervous system (PNS). Sensory neurons take in and communicate information about heat, cold, pressure, pain, position and more. In mammalian organisms, sensory neurons relay their information to the central nervous system (CNS) where it is then transmitted to the brain, where it can be further processed and acted upon. At the molecular level, sensory receptors located on the cell membrane of sensory neurons are responsible for the conversion of stimuli into electrical impulses.

Neurons are typically composed of a soma (i.e., cell body), a dendritic tree and an axon. The soma is the central part of the neuron and contains the cell nucleus. The dendritic tree is comprised of many cellular extensions or branches (individually called dendrites). The dendrites are where the majority of stimulatory inputs to the neuron occur. The axon is a finer, cable-like projection which can extend tens, hundreds, or even tens of thousands of times the diameter of the soma in length. The axon carries nerve signals away from the soma (and also carry some types of information back to it). Many neurons have only one axon, but their axon commonly exhibits extensive branching, thus enabling communication with many target cells. Sensory neurons have dendrites on both ends, connected by a long axon with a cell body in the middle. Axons have specialized structures at their ends that are used to release neurotransmitter chemicals and communicate with target neurons. In vertebrates, the axons of many neurons are sheathed in myelin, which is formed by either of two types of glial cells: (1) Schwann cells that ensheath peripheral neurons and (2) oligodendrocytes that insulate axons in the CNS. Along myelinated nerve fibers, gaps in the sheath known as nodes of Ranvier occur at evenly-spaced intervals and enable a very rapid mode of electrical impulse propagation called saltation.

The peripheral nervous system can be involved in a wide range of medical disorders with various pathophysiologies that are generally referred to as peripheral neuropathy. Despite the diverse array of medical disorders that cause peripheral neuropathies, peripheral nerves exhibit only a few distinct pathologic reactions to an insult or disease: (1) Wallerian degeneration, (2) axonal degeneration, and (3) segmental demyelination. The specific mechanisms by which the various disorders affecting peripheral nerve induce these pathologic changes are largely unknown. In Wallerian degeneration, the axon degenerates distal to a focal lesion that interrupts the continuity of the axon. This reaction often occurs in focal mononeuropathies that result from trauma or nerve infarction. Axonal degeneration, sometimes referred to as the “dying-back” phenomenon, is an active program of self-destruction that is observed in many physiological and pathological settings. Axonal degeneration typically occurs at the most distal extent of the axon. Axonal degenerative polyneuropathies are usually symmetric, and as the disorder progresses, the axons typically degenerate in a distal-to-proximal gradient. Axonal degeneration is the most common type of pathologic reaction in generalized polyneuropathies, and it is often attributed to a “metabolic” etiology. However, axonal degeneration may also occur as a secondary pathology associated with physical injuries and traumas to the PNS. Segmental demyelination refers to focal degeneration of the myelin sheath with sparing of the axon. This reaction can be seen in focal mononeuropathies but also in generalized sensorimotor or predominantly motor neuropathies. Acquired segmental demyelinating polyneuropathies are often immune-mediated or inflammatory in origin. However, segmental demyelination can also occur in some hereditary polyneuropathies.

The prognoses for peripheral nerve disorders associated with segmental demyelination are generally favorable because remyelination can be accomplished quickly thereby reestablishing normal conductivity of the axon and return of function. However, for those peripheral nerve disorders characterized by either Wallerian degeneration or axonal degeneration, prognosis is usually unfavorable due to the fact that the axon must regenerate and reinnervate muscle, the sensory organ, blood vessels, and other structures before clinical recovery is noted. Examples of peripheral neuropathies associated with axonal degeneration include diabetic sensory neuropathy, Alzheimer's disease, multiple sclerosis, distal symmetric polyneuropathy (clinically referred to as “DSP”) associated with HIV infections, and trauma-induced neuropathy.

In the PNS, on rare occasions, axons may successfully regenerate from damaged adult sensory neurons to form successful connections. However, although it is known that sensory axons may be regenerated under precisely controlled and manipulated laboratory conditions, in vivo scarring around physically traumatized or degenerated nerve tissue presents significant impediments to successful, i.e., functional axon regeneration. Those sensory axons that do regenerate, typically are not able to reform accurate connections with resident sensory axons, and because the CNS lacks plasticity to rewire in response to these “novel” (i.e., post maturation) connections, the functionality of regenerated sensory axons is unpredictable and tenuous.

### SUMMARY OF THE INVENTION

The exemplary embodiments of the present invention are directed to methods for producing purified individualized live adult sensory neural cells, apparatus and kits containing therein said purified individualized live adult sensory neural cells, and methods of use of said neural cells, apparatus and kits for rapid screening of small molecules for identification and selection of those small molecules that are capable of initiating and stimulating axon regeneration and growth from adult sensory neural cells.

One embodiment of the present invention is directed to an exemplary method for producing a suspension of purified individualized live adult sensory neural cells from a suitable laboratory animal.

An exemplary aspect of the method generally comprises removing the spinal column from a suitable laboratory animal, extracting dorsal root ganglia (DRG) from the spinal column and placing the extracted DRG into a suitable cell suspension medium, removing unnecessary satellite tissues and nerve fibers from the DRG, and then placing the cleaned DRG into a fresh cell suspension medium. The cleaned DRG is sequentially enzymatically digested first with a collagenase enzyme preparation followed by a trypsin enzyme preparation, and then washing the digested DRG tissues fetal bovine serum (FBS) to stop the enzymatic activity. After washing the digested DRG with fresh cell suspension medium to remove the FBS, the DRG neurons are separated from the cellular debris and tissues by column centrifugation, and then further purified. The purified DRG neurons are combined in a suitable plating medium with at least one growth factor selected from the group comprising nerve growth factor, glial cell line-derived growth factor, neurotrophin-3, and insulin. It is preferable that the purified DRG neurons are combined with all four growth factors.

Another embodiment of the present invention is directed to an exemplary method for rapidly screening small molecules to identify those small molecules that stimulate axon regeneration and outgrowth from adult sensory neurons.

One aspect of the method generally comprises first coating the well surfaces of a 96-well microplate with a suitable binding substrate such as exemplified by poly-DL-ornithine hydrobromide, and then overlaying the binding substrate with a laminin such as exemplified by mouse sarcoma cell-derived laminin. Then, an aliqout of the purified individualized live adult sensory neural cells produced as described above, is added to each well of the microplate, after which, a suitable dosage of a selected small molecule is dispensed into select wells while excluding a selected plurality of wells that serve as the control treatments. The 96-well microplate are then incubated under sterile conditions at 37° C. for at least twenty four hours after which, each well is morphometrically assessed to determine and record the extent of axon regeneration and outgrowth that occurred. Finally, the extent of axon regeneration and outgrowth occurring in the control treatments are compared with the axon regeneration and outgrowth occurring in the drug treatments, and are statistically analyzed to determine if any drug treatments stimulated axon regeneration relative to the control treatments.

According to another aspect, the small molecules are chemically derived candidates.

According to a yet another aspect, the small molecules are biologically derived candidates.

Another embodiment of the present invention is directed to an apparatus configured for large-volume rapid screening of small molecule candidates for assessment of their effects on axon regeneration and outgrowth from adult sensory neurons.

According to one aspect, the apparatus comprises a 96-well microplate wherein each well is coated with a suitable binding substrate such as exemplified by poly-DL-ornithine hydrobromide, and then overlaid with a laminin such as exemplified by mouse sarcoma cell-derived laminin. An aliquot of the purified individualized live adult sensory neural cells produced as described above, is added to each well of the microplate. The apparatus thus configured is suitable for dosing with candidate small molecules for subsequent incubation and examination of their effects on axon regeneration and outgrowth from the adult sensory neural cells.

According to one aspect, the apparatus is vacuum-sealable within a suitable plastic film.

According to another aspect, the apparatus is storable after vacuum sealing, at −70° C.

Another embodiment of the present invention is directed to a kit comprising a plurality of vacuum-sealed apparatus containing therein the purified individualized live adult sensory neural cells produced as described above. The kit additionally comprises instructions for preparing the apparatus for screening small molecules, adding dosages of small molecules to selected wells within the microplate, incubating the apparatus, morphometrically assessing the wells to determine the extent of axon regeneration and outgrowth, and statistically comparing the results from the control wells and the small molecule dosed wells.

A further embodiment of the present invention is directed to compositions configured for stimulating and promoting axon regeneration and outgrowth from adult sensory neurons. The compositions comprise a suitable carrier and at least one compound selected from the group exemplified by Aminoglutethimide, Aminopentoic acid, Baclofen, Caffeine, Chlorocresol, Dibucaine hydrochloride, Dihydrostreptomycin HCl, Ethopropazine HCL, Guaifenesin, Guanethidine sulphate, Hydrocortizone, Megastrol acetate, Methoxsalen, Phenazopyridine HCl, Sodium valproate, Bezafibrate, Aminopentanoic acid HCl, Pirenzepine HCl, Mesna, Metampicillin sodium, Atenolol, Ketoprofen, and the like.

### BRIEF DESCRIPTION OF THE DRAWINGS

The present invention will be described in conjunction with reference to the following drawings, in which:

FIG. 1 is a digitized photograph showing typical regenerated axons from isolated adult sensory neural cells produced with an exemplary method of the present invention;

FIG. 2 is graph showing the effects of growth factors on axon regeneration and elongation from adult sensory neurons;

FIG. 3 is a digitized photograph of a well with regenerated axons, partially showing part of a 30×30 μm grid laid over image for morphometric analysis;

FIG. 4 is a sample digitized photograph from a control well of another exemplary method of the present invention;

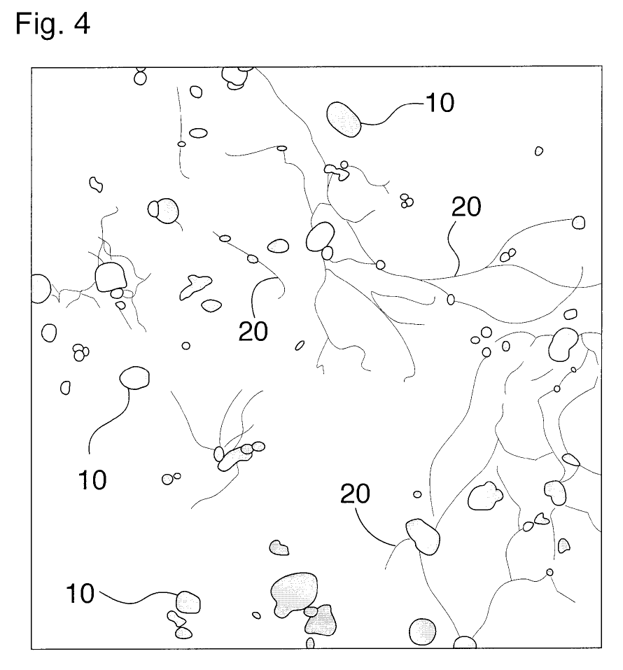

FIG. 5 is a sample digitized photograph from a drug-treated well from the same exemplary method as FIG. 4;

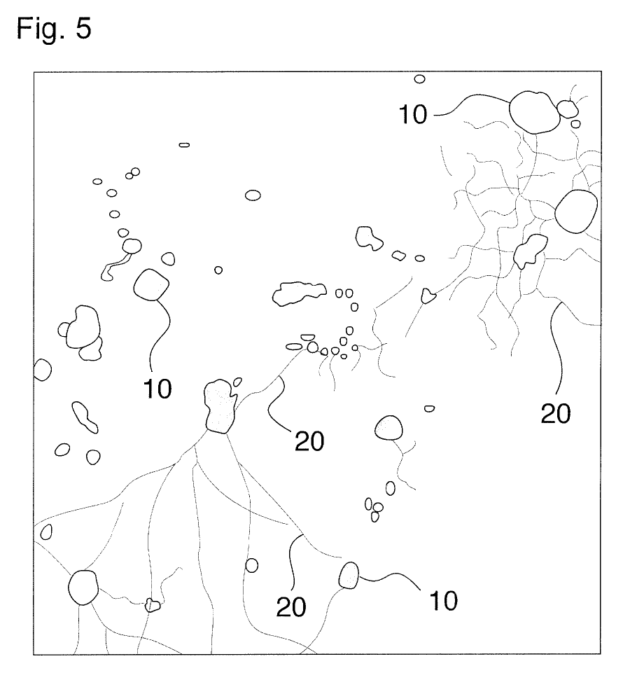

FIGS. 6(a)-(d) are charts showing the effects of selected small molecule compositions on outgrowths from adult neural cells isolated from streptozotocin diabetic rats. The values are means of triplicate samples ±SEM, * P≦0.001, ** P≦0.01; and

FIGS. 7(a)-(d) are charts showing the effects of selected small molecule compositions on outgrowths from adult neural cells isolated from Zucker diabetic fatty rats. The values are means of triplicate samples ±SEM, * P≦0.005 vs control (one-way ANOVA), ** P≦0.05 vs control, 0.1 μM and 1.0 μM doses (oneway ANOVA), and *** P≦0.05 vs control (t-Test).

### DETAILED DESCRIPTION OF THE INVENTION

The present invention discloses methods for producing individualized adult sensory neural cells, apparatus and kits containing the individualized adult sensory neural cells, and methods for using the apparatus and kits of the present invention for rapidly screening small molecules for assessment of their usefulness for initiation and stimulation of axon regeneration from adult sensory neurons. The present invention also discloses exemplary selections of drug compositions for stimulation of axon regeneration and outgrowth from adult sensory neural cells.

Exemplary methods for producing purified individualized live adult sensory neural cells are generally outlined in Example 1, while exemplary methods generally outlining their use for rapid screening of libraries comprising small molecule biological and chemical therapeutic agents are provided in Examples 2 through 4.

### EXAMPLE 1

### Tissue Dissociation and Cell Culture of Adult Dorsal Root Ganglion Neurons

### Specimen Handling: (Pre-Dissection)

A single adult male Sprague-Dawley rat weighing between 250 g-450 g was anesthetized by isofluorane induction. After no response to external stimuli, the rat was sacrificed by guillotine.

### Specimen Handling: (Dissection)

The rat spinal column was removed dorsally from the cervical area to the tail.

Dorsal root ganglia (DRG) were extracted from the spinal column using pattern #5 Dumont Student Quality thumb forceps (product # 91150-20 supplied by Fine Science Tools Inc. North Vancouver, B.C., Canada) and 3-inch Vannas style iris scissors (product # 4112 supplied by CDMV, St. Hyacinthe, Q.C., Canada). Excised DRG tissue was placed into a cell suspension medium comprising pre-warmed Hams F12 nutrient mix (GIBCO® Prod. # 21700 supplied by Invitrogen Corp., Burlington, Canada, L7P 1A1; GIBCO is a registered trademark of the Invitrogen Corp., Carlsbad, Calif., USA) supplemented with an antibiotic/antimycotic suspension stabilized and cell culture tested (Prod. # A5955 supplied by Sigma-Aldrich; (the stock solution which comprised of 10,000 units/ml penicillin G; 10 mg/ml streptomycin sulfate; and 25 μg/ml amphotericin B, was diluted 1:100 in Hams F12 nutrient mix), and a 1/100 dilution of a stock N₂ additive that comprised: (1) 0.1 mg ml⁻¹ transferrin (Prod. # T0523 supplied by Sigma-Aldrich Canada Ltd., Oakville, ON, Canada, L6H 6J8); (2) 20 nM progesterone (Prod. # P0130 supplied by Sigma-Aldrich); (3) 100 μM putrescine (Prod. # P7505 supplied by Sigma-Aldrich); (4) 30 nM sodium selenite (Prod. # S9133 supplied by Sigma-Aldrich); and (5) 10 mg ml⁻¹ fatty acid-free bovine serum albumin (BSA)(Prod. # A6003 supplied by Sigma-Aldrich); all at final concentrations in the cell suspension medium. Unnecessary satellite tissues and nerve fibers were cleaned from the DRG tissue immersed in the cell suspension medium at ambient room temperature using # 5 student forceps and a #10 scalpel blade under a Fisher Scientific Stereomaster® dissecting microscope (Stereomaster is a registered trademark of Fischer Scientific Co., LLC., Hampton, N.H., USA). The cleaned DRG tissue was placed into fresh cell suspension medium.

### Tissue and Cell Dissociation:

The cleaned DRG tissue was digested enzymatically to remove collagen fibers and proteinaceous material thereby releasing single DRG cells, as follows. First, the cleaned DRG tissue was placed into the cell suspension medium to which a collagenase enzyme preparation (Prod. # LS004194 supplied by Worthington Biochemical Corp., Lakewood, N.J., USA) was added to a final concentration of 0.125%, and then incubated for one hour at 37° C. in a sterile atmosphere comprising 5% CO₂. The DRG tissue was then transferred to a fresh collagenase enzyme-cell suspension medium and incubated for another hour, after which the collagenase-digested DRG tissue was aspirated and washed twice in cell suspension medium pre-warmed to ambient room temperature.

The washed collagenase-digested DRG tissue was then placed into cell suspension medium to which a trypsin enzyme preparation (Prod. # T5266 from porcine pancreas; supplied by Sigma-Aldrich) was added to a final concentration of 0.25%, and then incubated for twenty minutes at 37° C. in a sterile atmosphere comprising 5% CO₂. The trypsin action was stopped by the addition of 1 mL of pre-warmed HyClone fetal bovine serum albumin (FBS Prod. # SH30070.01 supplied by Thermo Fisher Scientific, Napean, ON, Canada). The enzyme-digested DRG tissue was then washed twice with cell suspension medium supplemented with 10% FBS to ensure deactivation of trypsin activity. The enzyme-digested DRG tissue was then washed twice with cell suspension medium to remove the FBS.

### Tissue and Cell Dissociation:

The enzyme-digested DRG tissue was taken up from the cell suspension medium into a borosilicate glass Pasteur pipette (note that a borosilicate glass pipette was used to minimize DRG tissue sticking to the pipette) and was triturated about 15 times to disrupt the DRG tissue and to disassociate individual cells, after which the large tissue fragments were allowed to settle out of the suspension. The suspended-cell-containing supernatent was removed and strained through a 70-μM mesh BD Falcon® cell strainer (Cat. #352350 supplied by BD Biosciences, Mississauga, ON, Canada; Falcon is a registered trademark of Becton Dickinson and Co. Corp., Franklin Lakes, N.J., USA), and the filtrate collected in a 15-mL BD Falcon® centrifuge tubes (Cat. # 352096 supplied by BD Biosciences). The filtrate, which contained the trituration-dispersed DRG neurons, was pelletized by centrifugation at 470 rpm for 10 minutes at ambient room temperature in an Eppendorf 5804-4 table top centrifuge (Eppendorf Canada, Mississauga, ON, Canada), thereby producing a soft pellet and a supernatent.

### Cell Purification:

The supernatent containing satellite cells and debris was aspirated and discarded. The soft pellet was resuspended in 200 μL of fresh cell suspension medium which was then layered over Ham's F12 medium (GIBCO® Prod. # 21700 supplied by Invitrogen Corp.) containing 15% BSA (Prod. # A-9205 supplied by Sigma-Aldrich) in a BD Falcons centrifuge tube (Cat. # 352096 supplied by BD Biosciences). The centrifuge tube was spun at 8100 rpm for 10 minutes at ambient room temperature in an Eppendorf 5804-4 table top centrifuge. The Ham's F12 medium containing 15% BSA served as a purification column for the supernatent and produced several upper layers containing dead cells, cellular debris and satellite layers over pellet of live neural cells. The upper layers were removed by aspiration with a Pasteur pipette. The cell pellet was resuspended in 1 mL of plating medium which comprised the cell suspension medium amended with 15 mM D-glucose (for a final concentation of 25 mM glucose). The 1 mL of suspended neural cells was then added to 19 mL of plating medium to make a 20-mL suspension of live neural cells.

### Cell Plating:

600 μL of the neural cell suspension was removed to a 1.5 mL Eppendorf centrifuge tube. The remaining 19.4 mL of cell suspension received the following growth factor supplements:
(1) Nerve Growth Factor (NGF) (Cat. # N1408 supplied by Sigma-Aldrich) added to a final concentration of 0.3 ng mL⁻¹;
(2) Glial Cell line-Derived Growth Factor (GDNF) (Cat. # G1777 supplied by Sigma-Aldrich) added to a final concentration of 5.0 ng mL⁻¹;
(3) Human recombinant Neurotrophin-3 (NT-3) (Cat. # N1905 supplied by Sigma-Aldrich) added to a final concentration of 1.0 ng mL⁻¹; and
(4) Insulin from porcine pancreas (Cat. # 15523 supplied by Sigma-Aldrich) added to a final concentration of 0.1 nM;

The well surfaces of a Nunc 96-well Optical Glass Bottomed CVG sterile plates (Prod. # 164590 supplied by VWR International, Edmonton AB, Canada) were coated with a binding substrate poly-DL-ornithine hydrobromide (Cat # P8638 supplied by Sigma-Aldrich) by first adding and then aspirating the substrate from the wells. Each well was then overlaid with 2 μg mL⁻¹ mouse sarcoma cell-derived laminin (GIBCO® Prod. # 23017-015 supplied by Invitrogen Corp.).

The 600-μL reserved neural cell suspension was separated into three 200-μL aliquots which were then each plated into a well. The growth-factor-amended neural cell suspension was then plated into the remaining 93 wells in 200-μL aliquots. DMSO (Cat. # D2650 supplied by Sigma-Aldrich) was added to the three control wells (i.e., containing neural cell suspension that was not amended with growth factor supplements). The 96-well plate was then stored in a direct-heat humidified incubator (Form a CO₂ Incubator supplied by Thermo Fisher Scientific) at 37° C. in a sterile atmosphere comprising 5% CO₂.

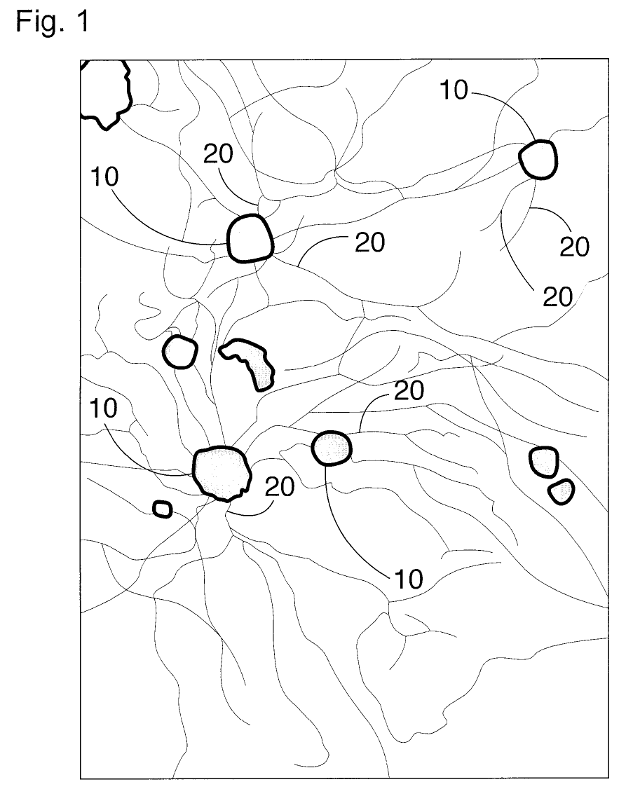

After a 24-h incubation, axon regeneration and growth were assessed and scored using the methods described by Gardiner et al. (2005, Molec. Cell. Neurosci. 28: 229-240). High resolution and high pixel density digitized images of neurons were acquired. FIG. 1 shows exemplary neural cells 10 and axons 20 that regenerated and grew out from the neural cells. Total axon outgrowth was determined by placing a 30×30 μm grid over the image and counting the total number of intercepts with the grid line. Total neurite lengths were determined by the summing the lengths of all the neurites produced by an individual neuron. This approach provides an accurate measure of total axon length either in relative units (number of crosspoints cell⁻¹) or in absolute units (μm cell⁻¹).

### EXAMPLE 2

### Effects of Selected Growth Factors on Axon Regeneration in Adult Neural Cell Suspensions

A 20.1-mL suspension of live neural cells was prepared from a single adult male Sprague-Dawley rat as described in Example 1. 600 μL of the neural cell suspension was removed to a 1.5 mL Eppendorf centrifuge tube. The remaining 19.5 mL of cell suspension were divided into three 3 6.5 mL aliquots. The first aliquot received a Low Dose of growth factors comprising 0.1 ng mL⁻¹ NGF, plus 1.0 ng mL⁻¹ GDNF, plus 0.1 ng mL⁻¹ NT-3, plus 0.01 nM insulin. The second aliquot received a Medium Dose of growth factors comprising 0.3 ng mL⁻¹ NGF, plus 5.0 ng mL⁻¹ GDNF, plus 1.0 ng mL⁻¹ NT-3, plus 0.1 nM insulin. The third aliquot received a Medium Dose of growth factors comprising 10.0 ng mL⁻¹ NGF, plus 50.0 ng mL⁻¹ GDNF, plus 50.0 ng mL⁻¹ NT-3, plus 10.0 nM insulin.

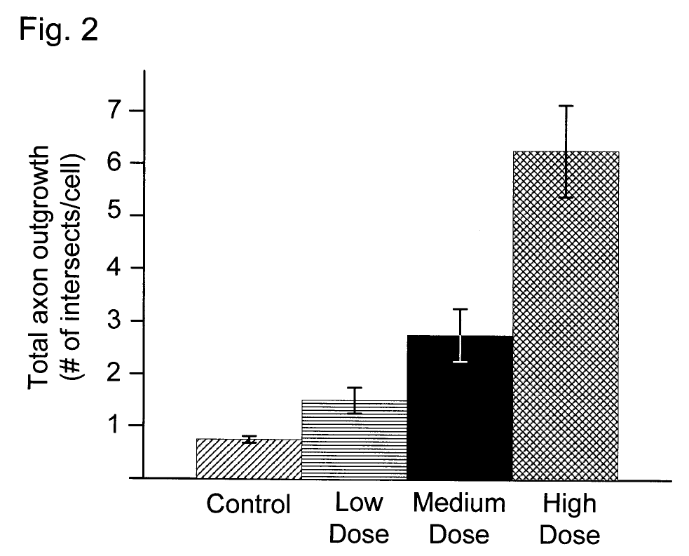

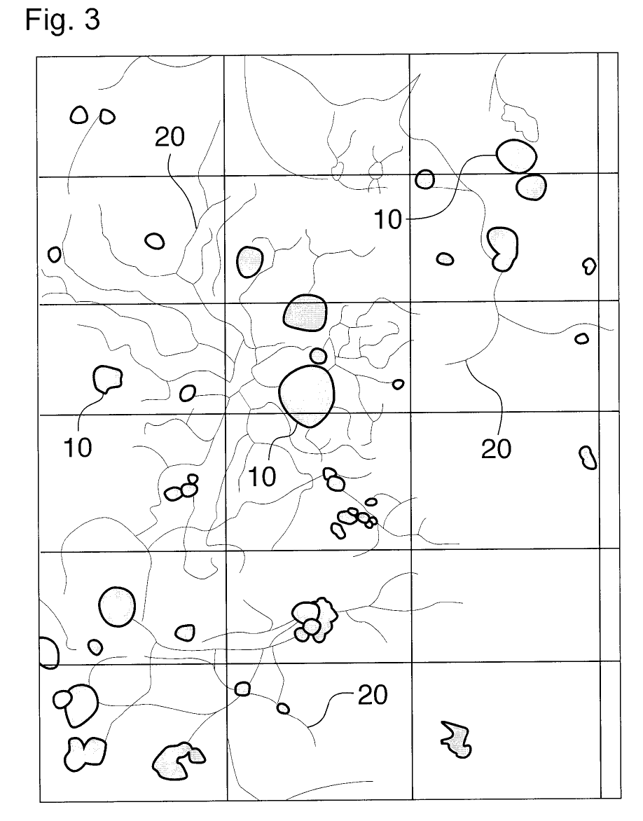

The well surfaces of a Nunc 96-well Optical Glass Bottomed CVG sterile plate were coated with a poly-DL-ornithine hydrobromide binding substrate and then overlaid with mouse sarcoma cell-derived laminin as described in Example 1. The 600-μL reserved neural cell suspension was separated into three 200-μL aliquots which were then each plated into a well. DMSO was added to the three control wells. The three growth-factor-amended neural cell suspensions were then successively plated into the remaining 93 wells in 200-μL aliquots. The 96-well plate was then stored in a direct-heat humidified incubator (Form a CO₂ Incubator supplied by Thermo Fisher Scientific) at 37° C. in a sterile atmosphere comprising 5% CO₂. After 48 hours, axon regeneration and growth were assessed and scored using the methods described Example 1. The effects of the increasing concentrations of growth factors on axon regeneration and growth from adult sensory neurons are shown in FIG. 2. FIG. 3 shows a portion of a 30×30 μm grid overlaid on a digitized image used to generate the data in FIG. 2.

### EXAMPLE 3

### Effects of Selected Small Molecules on Axon Regeneration in Adult Neural Cell Suspensions

A selection of drugs & bioactive compounds proven to be an innovative tool in drug discovery from the NIH-JDRF Custom Collection II was obtained in drug collection microplates from MicroSource Discovery Systems (Gaylordsville, Conn., USA) and stored at −20° C. The individual small molecule compounds were annotated for continuity in code series. Prior to use, a NINDS microplate was transferred to a 4° C. refrigerator for gradual thawing. About an hour before application, the NINDS microplate was transferred to a biosafety hood for thawing to be completed at ambient room temperatures while protected from light. Each of the drugs tested (stock concentrations were 10 mM) was diluted 1/100 in plating medium and then added to the neural cell suspensions in the 96-well plate to a final concentration of 10 μM (3 wells of neural cell suspension per drug type). The drug-treated neural cell suspensions were then incubated for 24 hrs at 37° C. in a sterile atmosphere comprising 5% CO₂ in a Form a direct-heat humidified incubator. The cell suspensions were then examined with a Zeiss LSM confocal microscope (Carl Zeiss Canada Ltd., Toronto, ON Canada) using brightfield illumation under 20× magnification and the effects of the various drugs on axon regeneration and growth were assessed and scored using the methods described by Gardiner et al. (2005, Molec. Cell. Neurosci. 28: 229-240). Total axon outgrowth was determined by placing a 30×30 μm grid over the image and counting the total number of intercepts with the grid line. This approach provides an accurate measure of total axon length either in relative units (number of crosspoints per cell) or in absolute units (total neurite length—the sum of the length of all the neurites produced by an individual neuron).

A total of 407 small molecule chemical drug formulations selected from the NINDS collection were screened in 15 separate assays. Twenty one of these drugs significantly stimulated axon regeneration and outgrowth from adult sensory neural cells. Their effects are summarized in Tables 1-9.

TABLE 1
Effects of Baclofen on axon regeneration
from adult sensory neurons

| | Control * | Baclofen ** | T-Test |
|---|---|---|---|
| Ave number of intersects per cell per | 4.5 ± 0.9 | 9.8 ± 2.2 | 0.026 |
| well | | | |

* average of 4 fields from 6 wells, i.e., a total of 24 fields ± SE
** average of 4 fields from 3 wells, i.e., a total of 12 fields ± SE

TABLE 2
Effects of Aminoglutethimide on axon regeneration
from adult sensory neurons

| | | Amino- | |
|---|---|---|---|
| | | glutethi- | |
| | Control * | mide ** | T-Test |
| Ave number of intersects per cell per | 2.4 ± 0.5 | 5.9 ± 2.0 | 0.070 |
| well | | | |

* average of 4 fields from 6 wells, i.e., a total of 24 fields ± SE
** average of 4 fields from 3 wells, i.e., a total of 12 fields ± SE

TABLE 3
Effects of selected drug molecules on axon
regeneration from adult sensory neurons

| | Ave number of | |
|---|---|---|
| | intersects per cell | |
| | per well | T-Test |
| Control * | 0.5 ± 0.2 | |
| Caffeine ** | 3.25 ± 1.6 | 0.042 |
| Chlorocresol ** | 2.8 ± 1.2 | 0.029 |
| Dibucaine hydrochloride ** | 2.0 ± 0.3 | 0.005 |
| Dihydrostreptomycin HCl ** | 2.4 ± 0.6 | 0.007 |
| Ethopropazine HCL ** | 4.4 ± 1.4 | 0.005 |

* average of 4 fields from 6 wells, i.e., a total of 24 fields ± SE
** average of 4 fields from 3 wells, i.e., a total of 12 fields ± SE

TABLE 4
Effects of Guanethidine sulphate on axon
regeneration from adult sensory neurons

| | | Guanethidine | |
|---|---|---|---|
| | Control * | sulphate ** | T-Test |
| Ave number of intersects per cell per | 2.8 ± 0.9 | 10.2 ± 3.7 | 0.018 |
| well | | | |

* average of 4 fields from 6 wells, i.e., a total of 24 fields ± SE
** average of 4 fields from 3 wells, i.e., a total of 12 fields ± SE

TABLE 5
Effects of Hydrocortizone on axon regeneration
from adult sensory neurons

| | | Hydrocorti- | |
|---|---|---|---|
| | Control * | zone ** | T-Test |
| Ave number of intersects per cell per | 4.3 ± 0.8 | 10.7 ± 1.7 | 0.005 |
| well | | | |

* average of 4 fields from 6 wells, i.e., a total of 24 fields ± SE
** average of 4 fields from 3 wells, i.e., a total of 12 fields ± SE

TABLE 6
Effects of selected drug molecules on axon
regeneration from adult sensory neurons

| | Ave number of | |
|---|---|---|
| | intersects per cell | |
| | per well | T-Test |
| Control * | 4.3 ± 1.1 | |
| Megastrol acetate ** | 9.2 ± 1.8 | 0.048 |
| Methoxsalen ** | 9.7 ± 2.0 | 0.037 |
| Phenazopyridine HCl ** | 10.2 ± 0.2 | 0.009 |

* average of 4 fields from 6 wells, i.e., a total of 24 fields ± SE
** average of 4 fields from 3 wells, i.e., a total of 12 fields ± SE

TABLE 7
Effects of Guaifenesin on axon regeneration
from adult sensory neurons

| | | Guaifene- | |
|---|---|---|---|
| | Control * | sin ** | T-Test |
| Ave number of intersects per cell per | 1.5 ± 0.4 | 5.0 ± 0.9 | 0.003 |
| well | | | |

* average of 4 fields from 6 wells, i.e., a total of 24 fields ± SE
** average of 4 fields from 3 wells, i.e., a total of 12 fields ± SE

TABLE 8
Effects of Sodium valproate on axon regeneration
from adult sensory neurons

| | | Sodium | |
|---|---|---|---|
| | Control * | valproate ** | T-Test |
| Ave number of intersects per cell per | 3.9 ± 0.5 | 8.5 ± 1.8 | 0.015 |
| well | | | |

* average of 4 fields from 6 wells, i.e., a total of 24 fields ± SE
** average of 4 fields from 3 wells, i.e., a total of 12 fields ± SE

TABLE 9
Effects of selected drug molecules on axon
regeneration from adult sensory neurons

| | Ave number of | |
|---|---|---|
| | intersects per cell | |
| | per well | T-Test |
| Control * | 1.9 ± 0.6 | |
| Bezafibrate ** | 6.9 ± 1.7 | 0.011 |
| Aminopentanoic acid HCl ** | 4.3 ± 0.4 | 0.044 |
| Pirenzepine HCl ** | 4.4 ± 0.7 | 0.047 |
| Mesna ** | 8.8 ± 3.9 | 0.040 |
| Metampicillin sodium ** | 6.7 ± 1.3 | 0.007 |
| Atenolol ** | 6.2 ± 0.2 | 0.003 |
| Ketoprofen ** | 7.9 ± 0.7 | 0.001 |

* average of 4 fields from 6 wells, i.e., a total of 24 fields ± SE
** average of 4 fields from 3 wells, i.e., a total of 12 fields ± SE

### EXAMPLE 4

### Effects of Small Molecules on Axon Regeneration in Adult Neural Cell Suspensions from Streptozoticin Diabetic Rats

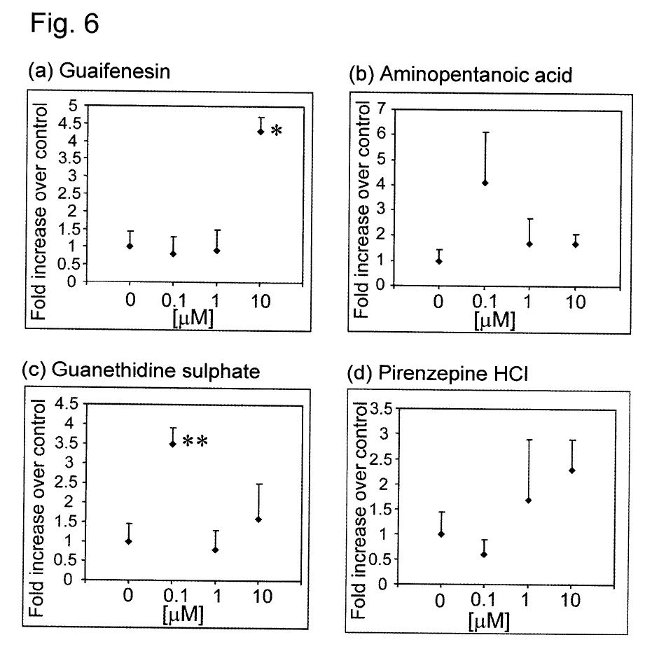

One-month-old Sprague-Dawley male rats were purchased from Charles River Laboratories, Inc. (Wilmington, Mass., USA) and cared for in an animal housing facility at the University of Manitoba (Winnipeg, MB, CA). The rats were made diabetic with onw intraperitoneal injection of 65 mg/kg streptozotocin (STZ; model of type 1 diabetes). The animals' blood-glucose levels were tested weekly, and the majority became hyperglycemic within one week. Confirmed hyperglycemic STZ rats were randomly selected at three months of diabetes and processed to produce adult neural cell suspensions as described in Example 1. The dose effects of four selected small molecule chemical drug formulations on the outgrowth of axons from individual cells in the cell suspensions were assessed by transferring an aliquot of an adult neural cell suspension to fresh cell suspension medium, in triplicate, containing 25 mM glucose amended with a selected dosage of a selected drug formulation. The four drug formulations assessed in this Example were Guaifenesin, Aminopentanoic acid, Guanethidine sulphate, and Pirenzepine HCl. The concentrations of each drug formulation assessed were 0 (control), 0.1 μM, 1.0 μM, and 10.0 μM. After a 24-h incubation, axon regeneration and growth were assessed and scored using the methods described in Example 1. A 0.1 μM concentration of Guanethidine sulphate and a 10.0 μM concentration of Guaifenesin significantly increased axon outgrowth from adult neural cells isolated from three-month-old STZ rats relative to the controls (FIGS. 6(c) and 6(a) respectively) while Aminopentanoic acid and Pirenzepine HCl did not have significant effects at the dosages tested in this example (FIGS. 6(b) and 6(d) respectively).

### EXAMPLE 5

### Effects of Small Molecules on Axon Regeneration in Adult Neural Cell Suspensions from Zucker Diabetic Fatty Rats

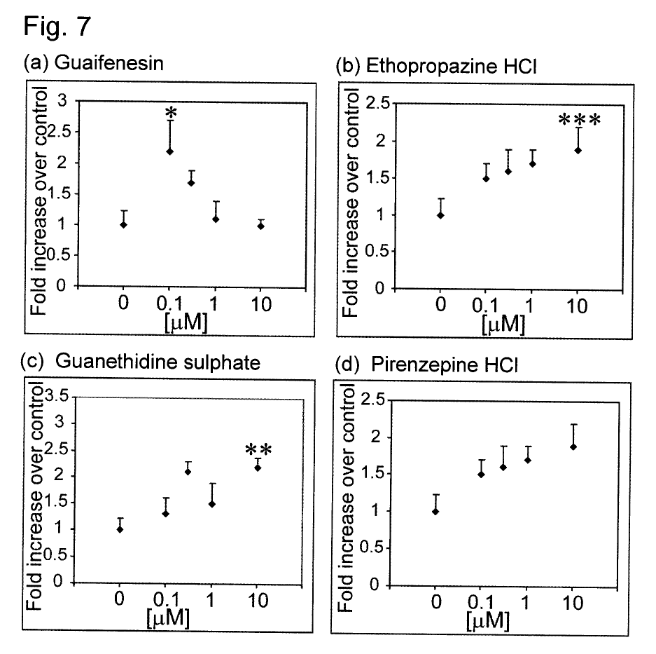

Four-week to six-week-old Zucker diabetic fatty rats (ZDF rats; model of type 2 diabetes) were purchased from Charles River Laboratories, Inc. (Wilmington, Mass., USA) and cared for in an animal housing facility at the University of Manitoba (Winnipeg, MB, CA). The animals' blood-glucose levels were tested weekly, and the majority became hyperglycemic at an age of about two months. The rats were maintained in a diabetic state for approximately four months. Confirmed 4-month hyperglycemic ZDF rats were randomly selected and processed to produce adult neural cell suspensions as described in Example 1. The dose effects of four selected small molecule chemical drug formulations on the outgrowth of axons from individual cells in the cell suspensions were assessed by transferring an aliquot of an adult neural cell suspension to fresh cell suspension medium, in triplicate, containing 25 mM glucose amended with a selected dosage of a selected drug formulation. The four drug formulations assessed in this Example were Guaifenesin, Ethopropazine HCl, Guanethidine sulphate, and Pirenzepine HCl. The concentrations of each drug formulation assessed were 0 (control), 0.1 μM, 1.0 μM, and 10.0 μM. After a 24-h incubation, axon regeneration and growth were assessed and scored using the methods described in Example 1. A 0.1 μM concentration of Guaifenesin, a 10.0 μM HCl concentration of Ethopropazine HCl, and a 10.0 μM concentration of Guanethidine sulphate each significantly increased axon outgrowth from adult neurals cells isolated from four-month old ZDF rats relative to the controls (FIGS. 7(a), 7(b) and 7(c) respectively) while Pirenzepine HCl did not have significant effects at the dosages tested in this example (FIG. 7(d)).

Those skilled in these arts will understand that the methods disclosed herein for producing purified individualized live adult sensory neural cells from rats can be easily modified for use with other types of suitable laboratory animals such as hamsters, mice, pigs and the like. Furthermore, those skilled in these arts will understand that the purified individualized live adult sensory neural cells of the present invention can be dispensed into suitable multi-well microplates, exemplified by 96-well microplates, that were previously coated with a suitable binding substrate and then overlaid with a suitable mouse sarcoma cell-derived laminin as described in Example 1, and then vacuum-sealed into sterile containers and stored at −70° C. for extended periods of time prior to use. Accordingly, such a multi-well microplate containing therein each well an aliquot of a purified individualized live adult sensory neural cells overlaid with a suitable binding substrate, such as laminin, comprises an apparatus of the present invention useful for rapid screening of small molecules for their usefulness in stimulating regeneration of axons and their outgrowth from said live neural cells. A kit of the present invention may comprise a plurality of vacuum-sealed multi-well microplates containing therein each well an aliquot of a purified individualized live adult sensory neural cells overlaid a suitable laminin overlaid a suitable binding substrate. The kit may contain a plurality of microplates wherein each microplate contains purified individualized live adult sensory neural cells prepared from the same mammalian species. Alternatively, the kit may contain a plurality of microplates wherein all of the micro plates contains purified individualized live adult sensory neural cells prepared from the same mammalian species, but each microplate contains selected different amounts of growth factors, e.g., low dose, medium dose and high dose concentrations. Alternatively, the kit may contain a plurality of microplates provided with purified individualized live adult sensory neural cells prepared from different mammalian species, but each microplate contains purified individualized live adult sensory neural cells prepared from the same mammalian species.

It is within the scope of the present invention to use the purified individualized live adult sensory neural cells produced from any suitable mammalian species using the exemplary methods disclosed herein for rapid screening of small molecule biological preparations using the methods disclosed therefore herein. Therefore, while this invention has been described with respect to the exemplary embodiments disclosed herein, it is to be understood that various alterations and modifications can be made to methods, apparatus and kits within the scope of this invention, which are limited only by the scope of the appended claims.

What is claimed is:

1. A method for producing purified individualized live adult sensory neural cells useful for screening small molecules for stimulation of axon regeneration, said method comprising the steps of:
removing a suitable laboratory animal's spinal column;
extracting dorsal root ganglia (DRG) from the spinal column and placing the extracted DRG into a suitable cell suspension medium;
cleaning the DRG by removing unnecessary satellite tissues and nerve fibers from the DRG, and placing the cleaned DRG into a fresh cell suspension medium;
sequentially enzymatically digesting the cleaned DRG with a collagenase enzyme preparation and a trypsin enzyme preparation thereby digesting the DRG and producing digested DRG tissues therefrom, and then washing the digested DRG tissues to stop the enzymatic activity;
separating individualized live adult sensory neural cells from the digested DRG tissues, and then purifying said individualized live adult sensory neural cells; and
combining the purified individualized live adult sensory neural cells in a suitable plating medium with at least one growth factor selected from the group consisting of nerve growth factor, glial cell line-derived growth factor, neurotrophin-3, and insulin to produce a purified individualized live adult sensory neural cell suspension.

2. A method for rapidly screening small molecules to identify small molecules that stimulate axon regeneration and outgrowth from adult sensory neurons, said method comprising the steps of:
coating the well surfaces of a multi-well microplate with a suitable binding substrate, and then overlaying the binding substrate with a laminin;
dispensing into each well an aliqout of the purified individualized live adult sensory neural cell suspension produced according to claim 1;
dispensing into a first selected well a suitable dosage of a first selected small molecule, and into at least a second selected well a suitable dosage of a second selected small molecule, while excluding a selected plurality of wells from said first and at least second dosages, said excluded wells serving as control treatments;
incubating the multi-well microplate under sterile conditions at about 37° C. for at least twenty four hours;
morphometrically assessing each well to assess and record the extent of axon regeneration and outgrowth that occurred from individual neural cells contained within said purified individualized live adult sensory neural cell suspension;
comparing the extent of axon regeneration and outgrowth occurring in the control treatments with the axon regeneration and outgrowth occurring in the wells receiving the first dosage and at least second dosage, and statistically determining if any of said dosages stimulated axon regeneration relative to the control treatments; and
using the statistical determination to select at least one small molecule for preparation therewith of a pharmaceutical composition configured for stimulating axon regeneration and outgrowth from live adult sensory neural cells.

3. A method according to claim 2, wherein the multi-well microplate is a 96-well microplate.

4. A method according to claim 2, wherein the small molecules are chemically derived small molecules.

5. A method according to claim 2, wherein the small molecules are biologically derived small molecules.

6. An apparatus configured for rapid screening of small molecules to identify small molecules that stimulate axon regeneration and outgrowth from live adult sensory neurons, said apparatus comprising a multi-well microplate wherein each well is coated with a suitable binding substrate and then overlaid with a suitable laminin, and each well is provided with an aliquot of a purified individualized live adult sensory neural cell suspension produced according to the method of claim 1.

7. An apparatus according to claim 6, wherein the multi-well microplate is a 96-well microplate.

8. An apparatus according to claim 6, wherein the apparatus is vacuum-sealable within a suitable plastics film.

9. An apparatus according to claim 6, wherein the apparatus is storable at a temperature selected from the range between −30° C. to −95° C.

10. An apparatus according to claim 6, wherein the apparatus is storable at a temperature of about −70° C.

11. A kit configured for rapid screening of small molecules for the identification of small molecules that stimulate axon regeneration and outgrowth from adult sensory neurons, said apparatus comprising:
a container containing therein a plurality of vacuum-sealed apparatus according to claim 6; and
instructions for preparing the apparatus for screening small molecules, adding dosages of small molecules to selected wells within the microplate, incubating the apparatus, morphometrically assessing the wells to determine the extent of axon regeneration and outgrowth, and statistically comparing the results from the control wells and the small molecule dosed wells.

12. A pharmaceutical composition configured for stimulating axon regeneration and outgrowth from adult sensory neurons, the pharmaceutical composition comprising:
a small molecule selected for stimulating axon regeneration and outgrowth from adult sensory neurons, said small molecule selected according to the method of claim 2; and
at least one of a pharmaceutically acceptable carrier, a pharmaceutically acceptable adjuvant, and a pharmaceutically acceptable excipient.

13. A pharmaceutical composition according to claim 12, wherein the small molecule is a biologically derived small molecule.

14. A pharmaceutical composition according to claim 12, wherein the small molecule is a chemically derived small molecule.

15. A pharmaceutical composition according to claim 12, wherein the small molecule is selected from a group consisting of Aminoglutethimide, Baclofen, Caffeine, Chlorocresol, Dibucaine hydrochloride, Dihydrostreptomycin HCl, Ethopropazine HCL, Guanethidine sulphate, Hydrocortizone, Megastrol acetate, Methoxsalen, Phenazopyridine HCl, Guaifenesin, Sodium valproate, Bezafibrate, Aminopentanoic acid HCl, Pirenzepine HCl, Mesna, Metampicillin sodium, Atenolol, and Ketoprofen.
